(19) United States
(12) Patent Application Publication
(10) Pub. No.: US 2006/0165394 A1
(43) Pub. Date: Jul. 27, 2006

(54) Process and apparatus for boiling add vaporizing multi-component fluids

(76) Inventor: Alexander I. Kalina, Hillsborough, CA (US)

Correspondence Address: ROBERT W STROZIER, P.L.L.C, PO BOX 429, BELLAIRE, TX, 77402-0429, US

(21) Appl. No.: 10/617,367

(22) Filed: Jul. 10, 2003

Related U.S. Application Data
(60) Provisional application No. 60/464,302, filed on Apr. 21, 2003.

(51) Int. Cl. A61L 9/03; A01G 13/06; A01M 13/00

(52) U.S. Cl. 392/386

(57) ABSTRACT

A new boiler or heat transfer apparatus is disclosed for use with multi-component working fluids which includes a vapor removal apparatus designed to maintain a substantial compositional identity between the boiling liquid and its vapor along a length of the apparatus resulting in the maintenance of substantially nucleate boiling along the entire length of the apparatus. Systems incorporating the apparatus and methods for making and using the apparatus are also disclosed.

### RELATED APPLICATIONS

This application claims provisional priority to U.S. Provisional Application Ser. No. 60/464302 and filing 21 Apr. 2003.

### BACKGROUND OF THE INVENTION

1. Field of the Invention

The present invention relates to an improved boiler apparatus, systems incorporating the boiler apparatus and to methods for making and using the boiler apparatus and systems incorporating the boiler apparatus.

More particularly, the present invention relates to an improved boiler apparatus, systems incorporating the boiler apparatus and to methods for making and using the boiler apparatus and systems incorporating the boiler apparatus, where the boiler apparatus includes a vapor removal unit that remove vapor as it boils so that the boiling throughout boiler's length remains substantially nucleate boiling.

2. Description of the Related Art

In several processes and especially in power systems using multi-component working fluids, it is necessary to completely vaporize such multi-component fluids. However, it is, in practice difficult to completely vaporize such multi-component fluid.

When a working fluid in the form of a saturated liquid is sent into a boiler, and the quantity of vapor in the stream of working fluid is relatively small, the boiling process is characterized as nucleate boiling. Nucleate boiling has a very high film heat transfer coefficient, but as vapor accumulates, a so-called crisis of boiling occurs. This crisis of boiling results in a drastic fall or reduction in the film heat transfer coefficient.

On the other hand, when a single-component fluid is vaporized, the liquid can be recycled within the heat exchanger and nucleate boiling can be sustained throughout the entire process. But, such an approach cannot be used with multi-component fluids, because the vapor produced will have a different composition (enriched by the low boiling component) than the remaining liquid resulting in a continuous composition profile across the heat exchanger with the concurrent crises of boiling.

Thus, if a multi-component fluid needs to be vaporized fully, the in a significant proportion of this vaporization process, i.e., inside the heat exchanger or boiler, nucleate boiling cannot be maintained. Thus, the film heat transfer coefficient in such a process is very low. This results in a very large increase in the required surface of the heat exchanger or boiler.

If complete vaporization of a multi-component working fluid has to be performed at high temperature, e.g., in a furnace of a power plant, then the inability of the process to maintain nucleate boiling inside heat transfer tubes of the furnace makes such a process technically very difficult.

When nucleate boiling is maintained, due to a high film heat transfer coefficient, the temperature of the metal of the heat transfer tubes is maintained close to the temperature of the boiling fluid, and as a result the tubes are protected from burn out. However, because in the process of direct vaporization of multi-component working fluids where nucleate boiling cannot be maintained, the heat transfer tubes can achieve unacceptably high temperatures resulting in tube damage or destruction.

Thus, there is a need in the art for process and apparatus for boiling and vaporization of multi-component fluids designed to achieve the production of vapor of the same composition as the composition of the initial multi-component liquid, and at the same time, to maintain a process of nucleate boiling in the heat transfer apparatus.

### SUMMARY OF THE INVENTION

The present invention provides an improved boiler or heat transfer apparatus including a vapor removal apparatus that removes vapor from a boiling working fluid so that substantially nucleate boiling occurs throughout the heat transfer apparatus and substantially full or complete vaporized of a multi-component working fluid occurs, where the multi-component working fluid comprises a low boiling component and a high boiling component.

The present invention also provides an improved vaporization apparatus for multi-component working fluids including a plurality of heat transfer apparatuses, each apparatus including a heat exchange unit and a vapor removal or collector unit, where the vapor collector units are adapted to maintain substantially nucleate boiling throughout each heat exchange unit and where the vaporization apparatus converts a liquid multi-component fluid feed having a given composition into a vapor stream having substantially the same composition.

The present invention provides a system for extracting heat from a heat source and converting a portion of the heat into a useable form of energy including a heat source stream, a multi-component working fluid, a vaporization apparatus of this invention, and a heat extraction system.

The present invention provides a method for vaporizing a liquid multi-component working fluid having a given composition into a vapor multi-component working fluid having substantially the same compositions, where the method includes the steps of feeding the liquid multi-component working fluid stream into an improved multi-component working fluid vaporization apparatus of this invention from a energy production facility, inputting a heat source stream from a heat source, outputting an spent heat source stream to the source and sending a vapor multi-component working fluid stream back to the energy production facility, where the liquid multi-component working fluid and the vapor multi-component working fluid have substantially the same composition and the vaporization apparatus maintains substantially nucleate boiling throughout all heat exchange units.

The present invention provides a methods for vaporizing a multi-component working fluid having a given composition including the steps feeding an input liquid multi-component working fluid stream having a given composition into a first heat transfer apparatus including a first heat exchange unit and a first vapor removal unit and transferring heat from a heat source to the input liquid multi-component working fluid stream to produce a first vapor stream having a richer composition than the input liquid stream and a first liquid stream having a higher temperature and a leaner composition than the input liquid stream. The first liquid stream is forwarded to a second heat transfer apparatus and a including a second heat exchange unit and a second vapor removal unit and transferring heat from the heat source to the first liquid stream to produce a second vapor stream having a richer composition than the first liquid stream and a second liquid stream having a higher temperature and a leaner composition than the first liquid stream. If there are only two heat transfer apparatuses, then the second liquid stream is forwarded to an upper feed port of a scrubber, while the first and second vapor streams can either be combined into to combined vapor stream and forwarded to a lower feed port of the scrubber or forwarded individually to different ports of the scrubber, where the different ports are located based on a temperature of each vapor stream, higher temperature vapor streams are fed at ports higher up a length of the scrubber and lower temperature vapor streams are fed at ports lower down the length of the scrubber. The second liquid stream is preferably sprayed into the scrubber. The second liquid stream and the vapor streams contact each other in a counter-flow arrangement to produce a final vapor stream having a composition substantially identical to the composition the input liquid stream and a remaining liquid stream that is combined with the first liquid stream prior to feeding into the second heat transfer apparatus. For systems having more than two heat transfer apparatuses, each heat transfer apparatus produces a liquid and vapor stream via heat from the heat source. Each liquid stream is forwarded to the next heat transfer apparatus, while the vapor streams are either combined or individually forwarded to the scrubber along with the last liquid stream from the last heat transfer apparatus. The vapor removal units associated with each heat transfer apparatus insure that substantially nucleate boiling occurs throughout each heat exchange unit.

### DESCRIPTION OF THE DRAWINGS

The invention can be better understood with reference to the following detailed description together with the appended illustrative drawings in which like elements are numbered the same:

FIG. 1A depicts a diagram of a preferred embodiment of a heat transfer apparatus of this invention having a vapor removal apparatus;

FIG. 1B depicts a diagram of another preferred embodiment of a heat transfer apparatus of this invention having a vapor removal apparatus;

FIG. 2A depicts a diagram of another preferred embodiment of a heat transfer apparatus of this invention having a vapor removal apparatus;

FIG. 2B depicts a diagram of another preferred embodiment of a heat transfer apparatus of this invention having a vapor removal apparatus; and

FIG. 3 depicts a diagram of heat extraction and useable energy production facility including a multi-component vaporization apparatus of this invention.

### DETAILED DESCRIPTION OF THE INVENTION

The inventors have found that a heat transfer apparatus can be constructed for substantially, fully vaporizing a working fluid comprising at least two components one component having a boiling point less than the other component, at least one low boiling component and at least one high boiling component, which includes a vapor removal system adapted to maintain substantially nucleate boiling in a boiling/vaporization zone of the apparatus.

The present invention broadly relates to an improved boiling apparatus for substantially completely vaporizing a multi-component fluid to obtain a desired vapor stream having a desired temperature and composition, where the boiling apparatus includes a plurality of heat transfer apparatuses and a scrubber, where each heat transfer apparatus comprises a heat exchanger, heat transfer loop or mixture thereof and a vapor removal apparatus. The removal of vapor at each heat transfer stage maintains nucleate boiling in each of the heat transfer apparatuses.

The present invention also broadly relates to a method for substantially maintaining nucleate boiling through each stage of a multi-stage boiling apparatus including the steps of feeding a multi-component stream into a plurality of heat transfer apparatuses, each heat transfer apparatus includes a vapor collectors or separator apparatus, where the apparatus allows substantially complete vaporization of the multi-component fluid while maintaining nucleate boiling throughout each heat transfer apparatus.

The working fluids to be vaporized in the inventions of this application are multi-component fluids that comprises a lower boiling point component fluid—the low-boiling component—and a higher boiling point component—the high-boiling component. Preferred working fluids include, without limitation, an ammonia-water mixture, a mixture of two or more hydrocarbons, a mixture of two or more freon, a mixture of hydrocarbons and freon, or the like. In general, the fluid can comprise mixtures of any number of compounds with favorable thermodynamic characteristics and solubility. In a particularly preferred embodiment, the fluid comprises a mixture of water and ammonia.

It should be recognized by an ordinary artisan that at those point in the systems of this invention were a stream is split into two or more sub-streams, the valves that effect such stream splitting are well known in the art and can be manually adjustable or are dynamically adjustable so that the splitting achieves the desired improvement in efficiency.

Suitable heat exchange units include, without limitation, heat exchangers, heat transfer loop, or any other unit that can transfer heat from a heat source to a working fluid stream. Suitable vapor removal units include, without limitation, vapor/liquid separators such as drums or separation tanks, vapor collector or any other unit that can remove a vapor from a mixed vapor-liquid stream.

The term substantially when used with a composition means that the composition to two streams differs by no more than 5% in each component, preferably, no more than 2% in each component, particularly, no more than 1% in each component and especially, no more than 0.5% in each component, with zero (identical streams) being the ultimate goal. The term substantially when used in conjunction with nucleate boiling means that no more than 10% of the boiling that occurs in the heat exchange units is non-nucleate boiling, preferably, no more than 5% of the boiling that occurs in the heat exchange units is non-nucleate boiling, particularly, no more than 2.5% of the boiling that occurs in the heat exchange units is non-nucleate boiling, especially, no more than 1% of the boiling that occurs in the heat exchange units is non-nucleate boiling, with the ultimate goal being 0% of the boiling that occurs in the heat exchange units is non-nucleate boiling.

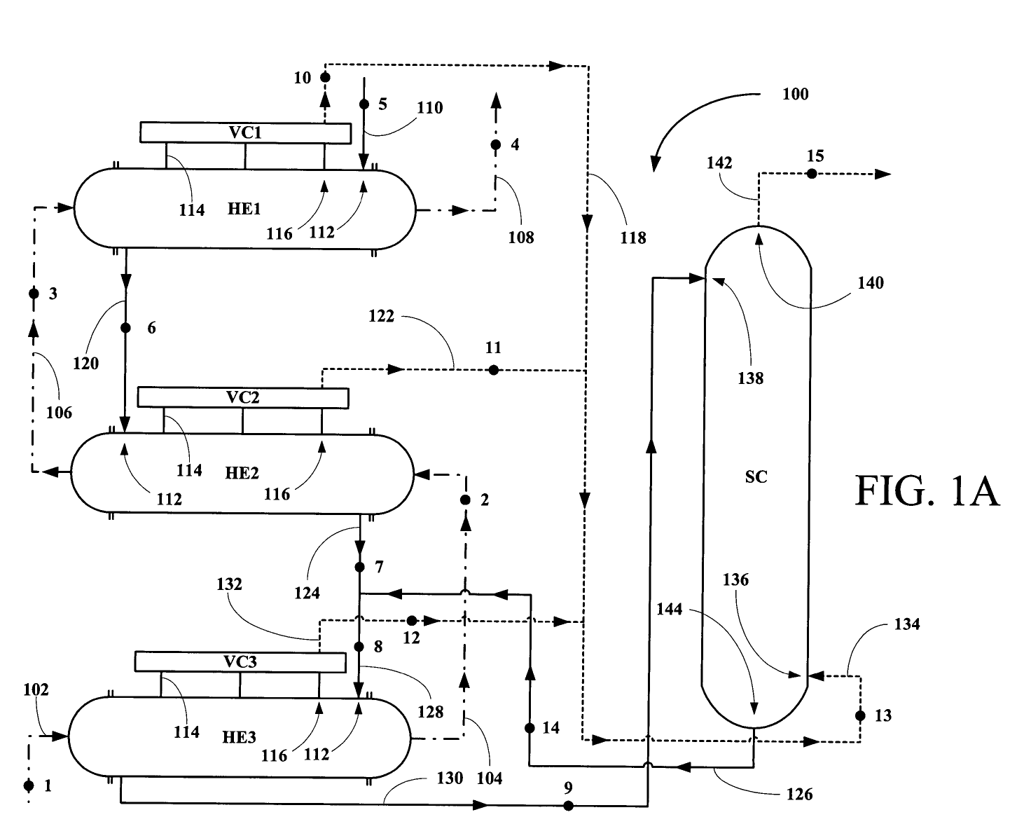

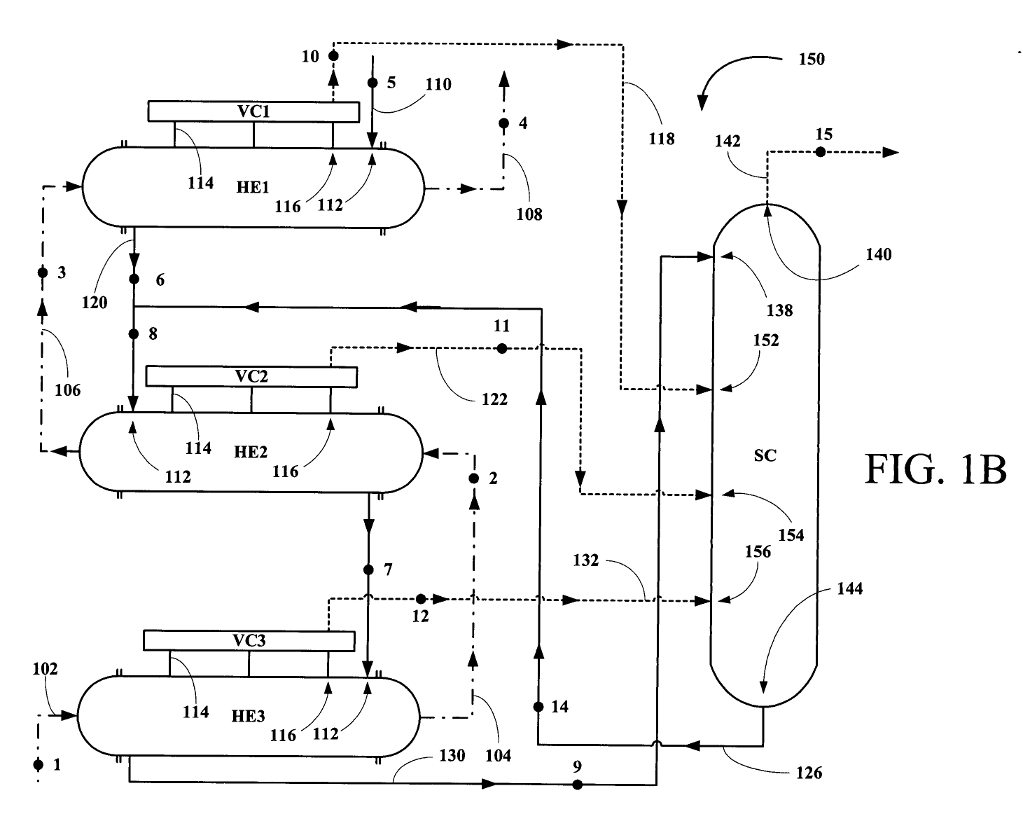

Referring now to FIG. 1A, a preferred embodiment of a heat transfer apparatus of this invention, generally 100, is shown to includes a heat source stream 102 having initial parameters as at a point 1, which is forwarded to a third heat exchanger HE3. The heat source stream 102 is preferably a hot vapor, liquid or mixed stream such as a geothermal brine stream, a stream from a power plant, or any other stream of hot fluid from any source. The stream 102 passes through heat transfer tubes (not shown) within the third heat exchanger HE3, where the stream 102 is cooled, releasing heat and leaves the third heat exchanger HE3 as a stream 104 having parameters as at a point 2. Thereafter, the stream 104 having the parameters as at the point 2 enters a second heat exchanger HE2, passes through it, and is further cooled, releasing further heat and leaves the second heat exchanger HE2 as a stream 106 having parameters at a point 3. Thereafter, the stream 106 having parameters as at the point 3 enters into a first heat exchanger HE1, passes through it, is yet further cooled, releasing yet further heat, and leaves the first heat exchanger HE1 as a stream 108 having parameters as at a point 4. Thus, the heat source stream 102 undergoes three heat transfers stages in heat exchangers HE1, HE2, and HE3. The heat from the four heat transfers stages is used to vaporize a multi-component stream 110 in the apparatus 100.

The multi-component working fluid stream 110 having parameters as at a point 5 corresponding to a state of saturated or slightly subcooled liquid, enters into the first heat exchanger HE1 on a shell side 112 thereof, passes through the first heat exchanger HE1, where it is heated by the heat source stream 106 having the parameters as at the point 3 to produce the heat source stream 108 having parameters as at the point 4. As the heat source stream 108 travels through the first heat exchanger HE1 heat is transferred to the working fluid stream 110 causing it to boil, releasing vapor along a length L of the first heat exchanger HE1. The produced vapor is constantly removed into a first vapor collector VC1 via a plurality of vent lines 114 at spaced apart locations 116 along the length L of the first heat exchanger HE1.

Because in the process of boiling, temperature changes along the length of the heat exchanger, the vapor produced in different parts of the heat exchanger will have different compositions. Thus, by removing the vapor at space apart locations along the length of the heat exchanger, the composition of the vapor can be maintained substantially the same as the boiling liquid allowing substantially nucleate boiling to occur along the length of the heat exchanger.

All of the vapor removed from the first heat exchanger HE1 is mixed in the first vapor collector VC1 and leaves the first vapor collector VC1 as a first vapor stream 118 having parameters as at a point 10. Meanwhile, the liquid leaving the first heat exchanger HE1 as a first liquid stream 120 having parameters as at a point 6 is hotter having been heated in the first heat exchanger HE1 and has a lower proportion of the low boiling component as compared to the liquid stream 110 having the parameters as at the point 5. Thereafter, the liquid stream 120 having the parameters as at the point 6 is sent into a shell side 112 of the second heat exchanger HE2, where it is further heated and boiled by heat released by the heat source stream 104 having parameters as at the point 2 as it passes through the second heat exchanger HE2 transferring heat to the liquid stream 120 to form the heat source stream 106 having the parameters of the point 3, sometime referred to as the 2-3 heating step. As in the first heat exchanger HE1, the vapor produced in the second heat exchanger HE2 is collected in a second vapor collector VC2 via a plurality of vent lines 114 at spaced apart locations 116 along the length of the second heat exchanger HE2, and leaves the second vapor collector VC2 as a second vapor stream 122 having parameters as at a point 11, while the liquid leaves the second heat exchanger HE2 as a second liquid stream 124 having parameters as at a point 7.

The second liquid stream 124 having the parameters as at the point 7 is then mixed with another stream of liquid 126 having parameters as at a point 14, as described below. In this embodiment of the present invention, a temperature and composition of the liquid stream 126 having the parameters 14 are substantially identical to a temperature and composition of the liquid stream 124 having parameters as at the point 7. As result of this mixing, a combined liquid stream 128 having parameters as at a point 8 is formed.

The liquid stream 128 having the parameters as at the point 8 then passes through into a shell side 112 of the third heat exchanger HE3, where it boils, producing vapor which is collected in a third vapor collector VC3. The unvaporized liquid leaves the third heat exchanger HE3, as a third liquid stream 130 having parameters as at a point 9, while the vapor produced in the third heat exchanger HE3 is collect in the third vapor collector VC3 and leaves the third vapor collector VC3 as a third vapor stream 132 having parameters as at a point 12.

The temperature of the third liquid stream 130 having the parameters as at the point 9 is a highest temperature achievable in this embodiment of the process of this invention. If the vapor collected in vapor collectors VC1, VC2 & VC3 was not removed from the liquid streams 110, 120 and 128 during heating, then the composition of the liquid stream 130 having the parameters as at the point 9 would be equal to the composition of the stream 110 having the parameters as at the point 5 and such a stream would have been fully vaporized at the temperature and pressure corresponding to the liquid stream 130 having the parameters as at the point 9. But because the vapor was removed as described above, the composition of the liquid stream 130 having parameters as at the point 9 is significantly leaner (i.e., has a lower concentration of the low-boiling component) than the stream 110. The state of the liquid stream 130 is a saturated liquid.

The vapor streams 118, 122, and 132 having parameters as at the points 10, 11 and 12, respectively, are combined into a combined vapor stream 134 having parameters as at a point 13. The stream 134 having the parameters as at the point 13 has a temperature which is substantially lower than the temperature of the third liquid stream 130 having the parameters as at the point 9. As was noted above, the liquid stream 130 having the parameters as at the point 9 is substantially leaner than the initial liquid stream 110 having the parameters as at the point 5. Conversely, the combined vapor stream 134 having the parameters as at the point 13 is significantly richer in the low-boiling component than the initial multi-component stream 110 having the parameters of at the point 5.

The intermediate removal of vapor has achieved the maintenance of nucleate boiling in all three heat exchangers HE1, HE2 and HE3. However, the produced vapor does not have the required temperature (which must be equal to the temperature of the composition of the third liquid stream 130 having the parameters as at the point 9) or the required composition (which must be equal to the composition of the initial multi-component stream 110 having the parameters as at the point 5) to achieve the complete vaporization of the initial multi-component liquid stream 110 having the parameters as at the point 5.

To accomplish these thermal and compositional requirements, the combined vapor stream 134 having the parameters as at the point 13 is sent into a lower part 136 of a vertical scrubber SC, while the liquid stream 130 having parameters as at the point 9 is sent into a upper part 138 of the scrubber SC. In the scrubber SC, the liquid stream 130 having the parameter as at point 9 is sprayed into the SC and the droplets fall down through the scrubber SC. Meanwhile, the combined vapor stream 134 having parameters as at the point 13 moves up through the scrubber SC. In such a counterflow of liquid and vapor arrangement, a very intensive heat and mass transfer occurs. The liquid, as a result of such a process, becomes cooler and richer, whereas the vapor becomes hotter and leaner. At a top 140 of the scrubber SC, the vapor from the stream 134 comes into equilibrium with the third liquid stream 130 having the parameters as at the point 9 acquiring the same temperature of the stream 130 having the parameters as at the point 9 and the same composition as the initial multi-component liquid stream 110 having the parameters as at the point 5.

This resulting vapor, leaves the top 140 of the SC as a fourth vapor stream 142 having the parameters as at the point 15. Meanwhile, the liquid is collected at the bottom of the scrubber SC, and leave a bottom 144 of the scrubber SC as the stream 126 having the parameters as at the point 14.

The temperature and composition of the SC liquid stream 126 having the parameters as at the point 14 depends on the flow rate of the third liquid stream 130 having the parameters as at the point 9, the larger the flow rate, the hotter and leaner the SC liquid stream 126 having parameters at the point 14. Therefore, it is possible to achieve a composition and temperature of the SC stream 126 having the parameters as at the point 14, which are practically the same as the composition and temperature of the second liquid stream 124 having the parameters of the point 7.

The SC stream of liquid 126 having the parameters as at the point 14 is combined with the third stream of liquid 124 having parameters as at the point 7 forming the combined liquid stream 128 having parameters as at the point 8 as described above.

Referring now to FIG. 1B, an alternate preferred embodiment of the apparatus of FIG. 1B, generally 150 is shown, where the vapor 118, 122 and 132 having the parameters of the points 10, 11 & 12, respectively, collected in the vapor collectors VC1, VC2 and VC3 are fed individually into the scrubber SC. In such a case, the individual vapor stream 118, 122 and 132 must be sent into different points along a height of the scrubber SC. The hottest stream 132 is fed into the SC at an upper feed port 152 of the SC, the middle temperature vapor stream 122 is fed into the SC at a middle feed port 154 of the SC, and the coldest stream 118 is fed into the SC at a lower feed port 156 of the scubber SC. Such a multi-point injection arrangement would increase the efficiency of the process in the scrubber SC, but would require more elaborate piping. In such a case, the liquid collected at the bottom 144 of the scrubber SC, will be cooler and, therefore, must be sent back into the system between HE1 and HE2 and combined with the stream 120 having the parameters as at the point 6 instead of between the heat exchanger HE2 and HE3 and combined with the stream 124 having the parameters of the point 7. The exact position of the ports 252, 254, and 256 will depend on the scrubber design, stream flow rates, stream compositions and other system criteria well known to ordinary artisans.

FIG. 1, shows the proposes system as including three heat exchangers, however, the proposed system will function with a minimum of two heat exchangers to as many heat exchangers as may be required for a given project. Preferably, the number of heat exchangers or heat exchange units are between 3 and 12 heat exchangers with between 3 and 8 being particularly preferred with between 3 and 6 being most preferred. One with ordinary experience in the art can design a specific embodiment of this system with a number of heat exchangers as required by circumstances. In the above embodiments, the vapor removal apparatus comprises a vapor collector associated with each heat exchanger.

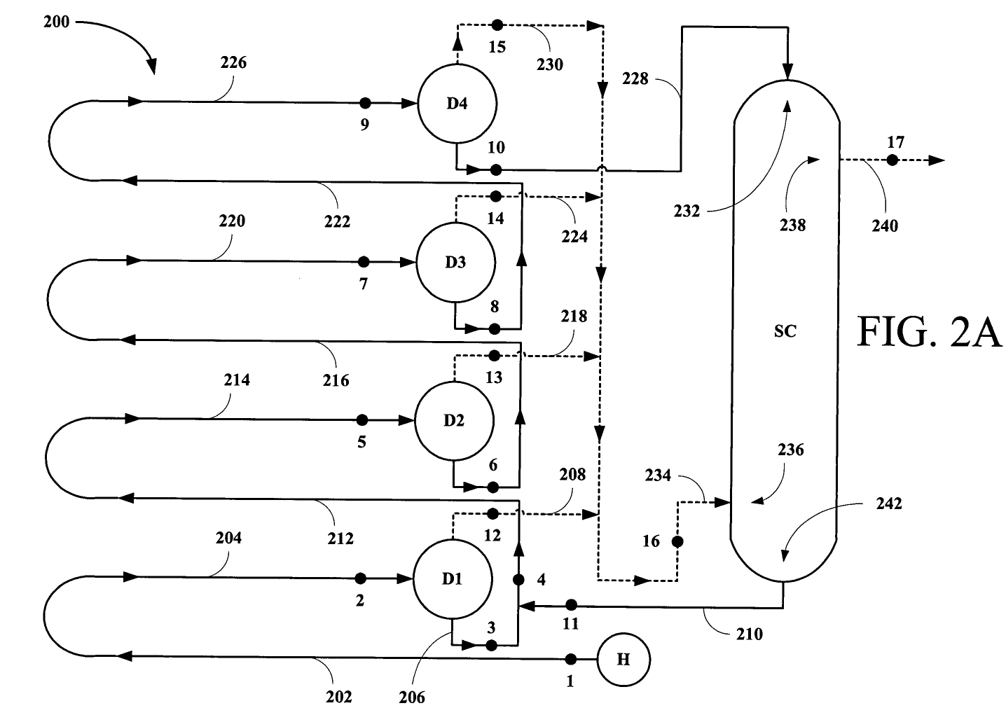

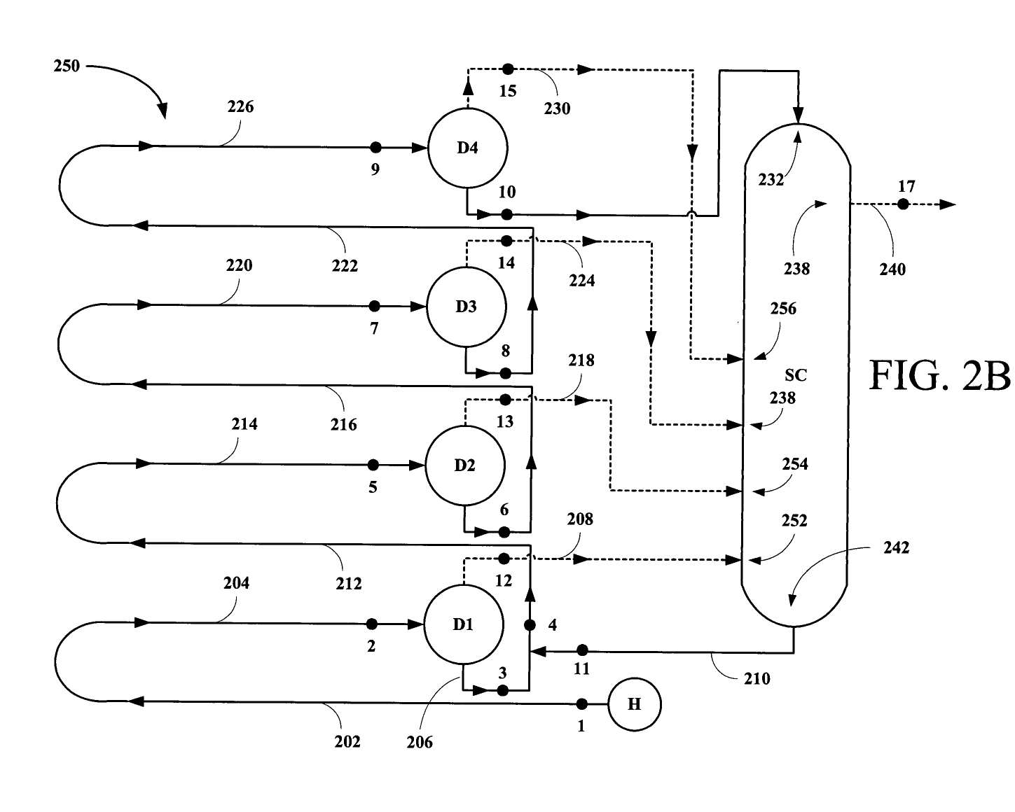

Variants of the proposed system designed for work at very high temperature (e.g., power plants such as nuclear or direct coal fired power systems) are shown in FIG. 2A&B. Referring now the FIG. 2A, another preferred system of this invention, generally 200, is shown to include four heat transfer loops HTL 1-4. A saturated liquid stream 202 to be vaporized and having parameters as at a point 1 is fed into the system from a header H, into the first heat transfer loop HTL1. After being partially vaporized in the loop HTL1, the saturated liquid stream 202 leaves as a first mixed stream 204 having parameters as at a point 2 and enters into a drum D1, where the first mixed stream 204 is separated into a first liquid stream 206 having parameters as at a point 3 and a first vapor 208 having parameters as at a point 12. The liquid stream 206 having the parameters as at the point 3 is combined with a SC liquid stream 210 having parameters as at point 11 from a scrubber SC to form a combined stream of liquid 212 having parameters as at a point 4.

The combined stream 212 having the parameter as at the point 4 is then sent into the second heat transfer loop HTL2, where it is partially vaporized producing a second mixed stream 214 having parameters as at a point 5. After being partially vaporized in the second loop HTL2, the second mixed stream 214 enters into a second drum D2, where the second mixed stream 214 is separated into a second liquid stream 216 having parameters as at a point 6 and a second vapor 218 having parameters as at a point 13.

The third liquid stream 216 having the parameters as at the point 6 is then sent into the third heat transfer loop HTL3, where it is partially vaporized producing a third mixed stream 220 having parameters as at a point 7. After being partially vaporized in the third loop HTL3, the third mixed stream 220 enters into a third drum D3, where the third mixed stream 220 is separated into a third liquid stream 222 having parameters as at a point 8 and a third vapor 224 having parameters as at a point 14.

The liquid stream 222 having the parameters as at the point 8 is then sent into the fourth heat transfer loop HTL4, where it is partially vaporized producing a fourth mixed stream 226 having parameters as at a point 9. After being partially vaporized in the fourth loop HTL4, the fourth mixed stream 226 enters into a fourth drum D4, where the stream 226 is separated into a fourth liquid stream 228 having parameters at a point 10 and a vapor 230 having parameters as at a point 15.

The fourth liquid stream 228 having parameters as at a point 10 is then forwarded to a top 232 of the SC. The fourth vapor stream 230 having the parameters as at the point 15, the third vapor stream 224 having the parameter as at the point 14, the second vapor stream 218 having the parameters as at the point 13, and the first vapor stream 208 having the parameters as at the point 12 are combined to from a combined vapor stream 234 having parameters as at a point 16.

Clearly, the processes in the heat transfer loops HTL2-4 are identical.

As in the case of the apparatus of FIGS. 1A&B, the combined vapor stream 234 does not have the required temperature (which most be equal to the temperature of the composition of the fourth liquid stream 228 having the parameters as at the point 10) or the required composition (which must be equal to the composition of the initial liquid stream 202 having the parameters as at the point 1) to achieve the complete vaporization of the liquid stream 202 having the parameters as at the point 1.

To accomplish this requirement, the combined vapor stream 234 having the parameters as at the point 16 is sent into a lower part 236 of the vertical scrubber SC, while the fourth liquid stream 228 having parameters as at the point 10 is sent into the top 232 of the scrubber SC. In the scrubber SC, the fourth liquid stream 228 having the parameter as at point 10 is sprayed and the droplets fall down through the scrubber SC. Meanwhile, the combined vapor stream 234 having parameters as at the point 16 moves up through the scrubber SC. In such a counterflow of liquid and vapor arrangement, a very intensive heat and mass transfer occurs. The liquid, as a result of such a process, becomes cooler and richer, whereas the vapor becomes hotter and leaner. Near the top 232 of the scrubber SC, the vapor stream 234 having the parameters as at the point 16 comes into equilibrium with the liquid stream 228 having the parameters as at the point 10 acquiring the same temperature as the stream 228 having the parameters as at the point 10 and the same composition as the stream 202 having the parameters as at the point 1. Thus, the system 200 has achieved the result of substantially complete or full vaporization of the multi-component stream 202 having the parameters as at the point 1.

This resulting vapor, leaves an upper port 238 of the SC as a stream 240 having the parameters as at the point 17. Meanwhile, the liquid is collected at a bottom 242 of the scrubber SC, and leave the scrubber SC as the stream 210 having the parameters as at the point 11.

The temperature and composition of the liquid stream 210 having the parameters as at the point 11 depends on the flow rate of the liquid stream 228 having the parameters as at the point 10, the larger the flow rate, the hotter and leaner the liquid stream 210 is at the point 11. Therefore, it is possible to achieve a composition and temperature of the stream 210 having the parameters as at the point 11, which are practically the same as the composition and temperature of the liquid stream 206 having the parameters of the point 3.

As a result of boiling, a hot liquid stream 228 having parameters as at the point 10, which is leaner than the initial liquid stream 202 having the parameter as at the point 1, and a stream 234 of vapor having parameters as at the point 16, which is cooler than the liquid stream 228 having the parameters as at the point 10 and richer than the liquid 202 having the parameters as at the point 1 is produced. These streams are then sent into the scrubber SC, which performs as described above and shown in FIGS. 1A&B to produce a fully vaporized stream 240 having a temperature substantially the same as the liquid stream 228 and a composition substantially the same as the stream 202.

As in FIG. 1B, the four vapor streams 208, 218, 224, and 230 can be fed separately to the scrubber SC to increase its efficiency, but at a cost of additional piping and valving. Referring now to FIG. 2B, another preferred embodiment of the system of FIG. 2A, generally 250 is shown, but with each individual vapor stream 208, 218, 224, or 230 being fed separately into the scrubber SC. The first vapor stream 208 having the lowest temperature is fed into the scrubber SC at a first and lowest vapor feed port 252. The second vapor stream 218 having a higher temperature is fed into the scrubber SC at a second vapor feed port 254. The third vapor stream 218 having a yet higher temperature is fed into the scrubber SC at a third vapor feed port 256. The fourth vapor stream 218 having the highest temperature is fed into the scrubber SC at a fourth and highest vapor feed port 258. The exact position of the ports 252, 254, 256 and 258 will depend on the scrubber design, stream flow rates, stream compositions and other system criteria well known to ordinary artisans.

As shown above, the system of this invention illustrated in FIGS. 1A&B allows maintenance of nucleate boiling because the heat exchangers are equipped with vapor collectors, where boiling occurs and at the same time, allows for the production of vapor having a desired temperature and composition. This result is achieved by recycling liquid through the chain of heat exchangers equipped with vapor collectors and the scubber. In the system of this invention illustrated in FIGS. 2A&B, maintenance of nucleate boiling in the heat transfer loops is achieve by equipping each heat transfer loop with a drum separator and in conjunction with the scruber allows boiling occurs and at the same time, allows for the production of a multi-component vapor having a desired temperature and composition.

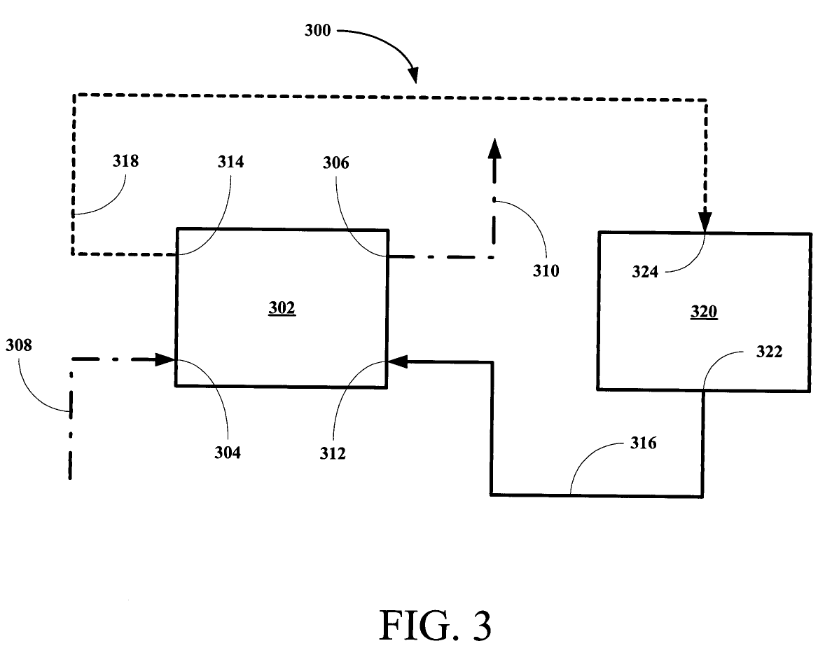

Referring now the FIG. 3, a preferred a heat extraction and energy production facility of this invention, generally 300, is shown to include a multi-component fluid vaporization apparatus of this invention 302. The apparatus 302 includes an heat source input 304 and an heat source output 306, where the input 304 inputs a heat source 308 shown here as an input heat source stream, but can be any other heat source and where the output 306 outputs a spent heat source 310 shown here as a spent heat source stream. Of course, if the heat source was focused sun light or other forms of electromagnetic radiation, then the input 304 would input light and the output 306 would output unused light.

The apparatus 302 also includes a liquid multi-component working fluid input 312 and a vapor multi-component working fluid output 314, where the liquid input 312 inputs an input liquid multi-component working fluid stream 316 and where the vapor output 314 outputs a final vapor multi-component working fluid stream 318. The liquid input stream 316 is output from an energy conversion unit 320 through a conversion unit liquid output 322, while the final vapor stream 318 is input to the energy convention unit 320 through a conversion unit vapor input 324. The energy conversion unit 320 extracts thermal energy from the final vapor stream 318 to produce the input liquid stream 316 and useable energy such as electrical energy or the like. Such energy conversion units can include any energy conversion unit known in the art including those described in U.S. Pat. Nos. 4,346,561; 4,489,563; 4,548,043; 4,586,340; 4,604,867; 4,674,285; 4,732,005;4,763,480; 4,899,545; 4,982,568; 5,029,444; 5,095,708; 5,440,882; 5,450,821; 5,572,871; 5,588,298; 5,603,218; 5,649,426; 5,754,613; 5,822,990; 5,950,433; 5,953,918; and 6,347,520; in co-pending U.S. patent application Ser. Nos. 10/242,301 filed 12 Sep. 2002; 10/252,744 filed 23 Sep. 2002; 10/320,345 filed 16 Dec. 2002, and 10/357,328 filed 03 Feb. 2003, incorporated herein by reference.

Thus, the processes and apparatuses (systems) provide for the full vaporization of multi-component fluids, the maintenance of high heat transfer coefficients in the boilers, and the protection of the boiler tubes from overheating in high temperature boilers or other higher temperature heat transfer systems.

All references cited herein are incorporated herein by reference. While this invention has been described fully and completely, it should be understood that, within the scope of the appended claims, the invention may be practiced otherwise than as specifically described. Although the invention has been disclosed with reference to its preferred embodiments, from reading this description those of skill in the art may appreciate changes and modification that may be made which do not depart from the scope and spirit of the invention as described above and claimed hereafter.

We claim:

1. A vaporization apparatus for multi-component working fluids comprising:
a plurality of n heat transfer apparatuses arranged in series, each heat transfer apparatus includes:
a heat exchange unit;
a vapor removal unit;
a liquid multi-component working fluid input;
a liquid multi-component working fluid output; and
a vapor multi-component working fluid output in fluid communication with the heat exchange unit; and
a scrubber,
where an input liquid multi-component working fluid stream is fed into the liquid input of the first heat transfer apparatus, each heat transfer apparatus produces a liquid stream and a vapor stream, the first n-1 liquid streams are forwarded to the next heat transfer apparatus in the series, the n^{th} liquid stream and the vapor streams are forwarded to the scrubber to produce a vapor multi-component having a substantially identical composition as the input liquid stream and where the vapor removal units are adapted to maintain substantially nucleate boiling throughout each heat exchange unit and where n has a numeric value of at least 2.

2. The vaporization apparatus of claim 1, wherein n has a numeric value between 3 and 12.

3. The vaporization apparatus of claim 1, wherein n has a numeric value between 3 and 8.

4. The vaporization apparatus of claim 1, wherein n has a numeric value between 3 and 6.

5. The vaporization apparatus of claim 1, wherein the multi-component fluid comprises a low-boiling component a high-boiling component.

6. The vaporization apparatus of claim 1, wherein the multi-component fluid is selected from the group consisting of an ammonia-water mixture, a mixture of at least two hydrocarbons, a mixture of at least two freon, a mixture of at least one hydrocarbon and at least one freon.

7. The vaporization apparatus of claim 1, wherein the multi-component fluid comprises an ammonia-water mixture.

8. The vaporization apparatus of claim 1, wherein the heat exchange units are selected from the group consisting of a heat exchanger and a heat transfer loop.

9. The vaporization apparatus of claim 1, wherein the vapor removal units are selected from a vapor collector and a vapor-liquid separation drum or tank.

10. A system for extracting heat from a heat source and converting a portion of the heat into a useable form of energy comprising:
a vaporization apparatus of claim 1-9, and
a heat extraction apparatus,
where heat from a heat source stream is transferred to a liquid multi-component working fluid stream having a given composition in the vaporization apparatus to produce a vapor multi-component working fluid stream having a substantially identical composition and where thermal energy transferred from the heat source stream to the vapor multi-component working fluid stream is converted into a more useable form of energy in the heat extraction apparatus.

11. A method for vaporizing a liquid multi-component working fluid comprising the steps of:
feeding a liquid multi-component working fluid stream into a multi-component working fluid vaporization apparatus of claims 1-9 from a energy production facility,
inputting heat from a heat source into the multi-component working fluid vaporization apparatus,
transferring the heat from the heat source to the liquid multi-component working fluid stream to produce a vapor multi-component working fluid stream; and
sending the vapor multi-component working fluid stream back to the energy production facility,
where the liquid multi-component working fluid and the vapor multi-component working fluid have substantially the same composition and the vaporization apparatus maintains substantially nucleate boiling throughout all heat exchange units having a given composition into a vapor multi-component working fluid having substantially the same composition, where the method

12. The method of claim 11, wherein the inputting step comprises:
inputting a heat source stream to the multi-component working fluid vaporization apparatus and
the method further comprising the step of:
outputting an spent heat source stream to the source and

13. A methods for vaporizing a multi-component working fluid comprising the steps:
feeding an input liquid multi-component working fluid stream having a given composition into an n^{th} heat transfer apparatus comprising an nth heat exchange unit and an n^{th} vapor removal unit;
transferring heat from a heat source in the nth heat exchange unit to the input liquid multi-component working fluid stream, where the heat causes a portion of the input liquid multi-component working fluid stream to boil;
removing vapor formed during the boiling via the n^{th} vapor removal unit to form an n^{th} vapor stream having a richer composition than the input liquid stream and an n^{th} liquid stream having a higher temperature and a leaner composition than the input liquid stream;
forwarding the n^{th} liquid stream to an n-1^{th} heat transfer apparatus comprising an n-1^{th} heat exchange unit and an n-1^{th} vapor removal unit;
transferring heat from the heat source in the n-i th heat exchange unit to the n^{th} liquid stream, where the heat causes a portion of the n^{th} liquid stream to boil;
removing vapor formed during the boiling via the n-1^{th} vapor removal unit to form an n-1^{th} vapor stream having a richer composition than the n^{th} liquid stream and an n-1^{th} liquid stream having a higher temperature and a leaner composition than the n^{th} liquid stream;
repeating the forwarding, transferring and removing step, while decrementing the counter by 1 until the counter has a numeric value of 1;
forwarding the 1^{st} liquid stream formed in the 1^{st} removing step and all of the vapor streams to a scrubber;
equilibrating the 1^{st} liquid stream and the vapor streams in the scrubber to produce a vapor multi-component working fluid stream having a composition substantially identical to the composition of input liquid multi-component working fluid stream and a remaining liquid stream; and
combining the remaining liquid stream from the scrubber with one of the liquid stream prior to forwarding that liquid stream to the next heat transfer apparatus, where that liquid stream has a temperature and composition that most closely matches a temperature and composition of the remaining liquid stream,
where vapor removal units associated with each heat transfer apparatus insure that substantially nucleate boiling occurs throughout each heat exchange unit.

14. The method of claim 13, wherein n is at least 2.

15. The method of claim 13, wherein n has a numeric value between 3 and 12.

16. The method of claim 13, wherein n has a numeric value between 3 and 8.

17. The method of claim 13, wherein n has a numeric value between 3 and 6.

18. The method of claim 13, wherein the multi-component fluid comprises a low-boiling component a high-boiling component.

19. The method of claim 13, wherein the multi-component fluid is selected from the group consisting of an ammonia-water mixture, a mixture of at least two hydrocarbons, a mixture of at least two freon, a mixture of at least one hydrocarbon and at least one freon.

20. The method of claim 13, wherein the multi-component fluid comprises an ammonia-water mixture.

21. The method of claim 13 wherein the heat exchange units are s elected from the group consisting of a heat exchanger and a heat transfer loop.

22. The method of claim 13, wherein the vapor removal units are selected from a vapor collector and a vapor-liquid separation drum or tank.
